(19) United States
(12) Patent Application Publication
(10) Pub. No.: US 2002/0168383 A1
(43) Pub. Date: Nov. 14, 2002

(54) Modifications of antigens by the introduction of aldehyde groups and their use in enhancing the immune response

(75) Inventors: Douglas T. Fearon, Cambridge (GB); Michael Allison, Cambridge (GB)

Correspondence Address: NIXON & VANDERHYE P.C., 1100 North Glebe Road, 8th Floor, Arlington, VA, 22201-4714, US

(73) Assignee: Cambridge University Technical Services Limited

(21) Appl. No.: 09/994,858

(22) Filed: Nov. 28, 2001

Related U.S. Application Data
(63) Continuation of application No. 09/673,725, filed on Oct. 20, 2000.

(30) Foreign Application Priority Data
Apr. 21, 1998 (GB) 9808485.8
Apr. 21, 1999 (GB) PCT/GB99/01206

(51) Int. Cl. A61K 39/12; C07K 17/00; C07K 14/00; C07K 1/00; A61K 39/002; A61K 39/00; A61K 39/02

(52) U.S. Cl. 424/204.1; 424/234.1; 424/265.1; 424/274.1; 530/350; 530/806

(57) ABSTRACT

The invention provides a method of producing an immune response in a mammal to an antigen which comprises modifying said antigen by introducing an alkyl aldehyde group into said antigen and introducing said modified antigen into the mammal. Periodate or glycolaldhyde may be used as the agent to introduce the aldehyde groups.

### FIELD OF THE INVENTION

The present invention concerns modifications of T-dependent antigens to increase their immunogenicity.

### BACKGROUND TO THE INVENTION

Vaccination is an effective means of combatting infectious disease. Traditionally, vaccines were based on attenuated, killed or fragmented microorganisms, and such vaccines are still in use at the present time. In recent years, the recombinant proteins and synthetic peptides have also been developed for vaccines.

To be effective, most vaccines have to be administrated with an adjuvant. Adjuvants increase the immune response of the body to a vaccine. Experimentally, many adjuvants exist. These include Freunds Complete Adjuvant (CFA; a mixture of killed M. tuberculosis, paraffin oil and mannide monooleate), aluminium salts (alum), surfactants, liposomes, saponins and adjuvant peptides. To date however, only alum is approved for human use and this is generally not as effective as some of the others, particularly CFA. There remains a need to improve the ability of vaccines to stimulate a protective immune response.

Zheng-B et al (1992, Science 256: 1560-1563) have investigated the recognition that takes place between antigen presenting cells (APCs) and T helper (T_{H}) lymphocytes. They note that in addition to the recognition which takes place between the T_{H} cells and the complex of antigen and MHC class II molecules, accessory interactions occur, including the carbonyl-amino condensation (Schiff base formation) between cell surface ligands. It was shown that co-injection of galactose oxidase with antigen promoted the immune response to that antigen. It was proposed that the formation of aldehydes on lymphocytes by galactose oxidase (after removal of sialic acid with neuraminidase) was the basis for the enhancement, and that galactose oxidase might be used in combination with conventional adjuvants.

Rhodes-J et al (1995, Nature 377: 71-75) reports a related study showing that administering Schiff-base-forming drugs to mice enhances several measures of immune responses.

Soltysik-S et al, (1995, Vaccine 13: 1403-1410), show that the ability of the sapcnin QS-21 to serve as an adjuvant is dependent on its single alehyde.

Apostolopoulos-V et al (1995, Proc. Natl. Acad. Sci., USA 92: 10128-10132) report that creating aldehydes on an antigen by coupling the antigen to periodate-oxidized mannan enhances its ability to elicit cytotoxic T cells and a Th1 response. The authors found a suppression of the antibody response, which was alleviated by conversion of the aldehydes on the mannan to —OH groups with borohydride.

Disclosure of the Invention

We have now found that modification of an antigen in order to introduce aldehyde groups to the antigen is effective in enhancing the immunogenicity of the antigen. Our data indicate that this modification alone renders an antigen more immunogenic than administering the unmodified antigen with alum alone.

We have observed the effect when oxidising the carbohydrates of glycosylated antigens. While not wishing to be bound by any one particular theory, we believe that this results in the generation of aldehydes by cleavage of the carbon-carbon bonds between vicinal hydroxyls of the carbohydrate.

We have also observed an enhancement by introducing aldehyde groups directly to a protein antigen by reaction with glycolaldehyde.

It is believed that the activity of the aldehydes may be related to their ability to form Schiff bases with epsilon amino groups on lysines of other proteins, present in the APC-T_{H} complex or elsewhere.

Thus in one aspect, a method is presented by which the immunogenicity of T-decendent antigens can be increased. This method involves the introduction of aldehydes on the antigen, for example by the oxidation with periodate of carbohydrate that is covalently associated with the antigen or by treatment of the antigen with an oxidising agent capable of introducing an alkyl aldehyde moiety in the antigen. The carbohydrate may be biosynthetically incorporated in the protein, as though N-linked oligosaccharides, or may be attached post-biosynthetically by a chemical process. The enhanced immunogenicity of the modified antigen is manifested both by increased antibody titre and priming of T cells. The enhancement in immunogenicity is restricted to the antigen that has been modified. The effect on immunogenicity remains after adsorption of modified antigen to alum. In order to introduce an aldehydes directly to an antigen we have used treatment with glycolaldehyde which has increased the immunogenicity of the protein.

A surprising aspect of the present invention is that our treatments result in enhanced antibody responses indicative of both a T_{H}1 and T_{H}2 response. Use of our modified antigens result in IgG1 antibody production, characteristic of a T_{H}2 response as well as IgG2a production, characteristic of a T_{H}1 response. This is in contrast to the results of Apostopoulos et al, who used oxidised mannan as an adjuvant, which was then reacted with a protein antigen (five VNTR repeats of the protein mucin). In contrast to the present invention, Apostopoulous et al report that reduction of the oxidised mannan-protein conjugate increases a T_{H}2 response.

The present invention thus provides a method of producing an immune response in a mammal to an antigen which comprises modifying said antigen by introducing an alkyl aldehyde group into said antigen and introducing said modified antigen into the mammal. The antigen may be a glycosylated polypeptide. In this case, a preferred means of modifying the antigen is by oxidation with sodium periodate. Alternatively, the antigen, whether or not it is glycosylated, may be modified by treatment with glycolaldehyde.

In another asnect, said antigen is introduced into the mammal together with an adjuvant.

The antigen may be any antiaen to which an immune response is desired. Such antigens particularly include bacterial, viral, fugal, parasitic and tumour antigens.

In another aspect, the invention provides novel compositions which comprise (i) an antiaen which has been modified by introduction of an alkyl aldehyde group, (ii) an adjuvant, and (iii) a pharmaceuticaliy acceptable diluent or carrier.

In a further aspect, the invention provides an antigen which has been modified by introduction of an alkyl aldehyde group for use in a method of treatment of the human or animal body, and further the use of an antigen which has been modified form by introduction of an alkyl aldehyde group for the manufacture of a medicament for immunotherapy of a human or animal subject.

### DESCRIPTION OF THE DRAWINGS

FIG. 1 shows IgG1 and IgG2 antibody responses in mice immunized with ovalbumin modified in accordance with the invention.

FIG. 2 shows antibody responses in different mouse strains.

FIG. 3 shows antibody responses to modified OVA and unmodified HEL.

FIG. 4 shows recall lymph node proliferation after in vivo priming with 5 μg OVA antigen.

FIG. 5 shows IgG1 and IgG2 anti-OVA responses.

FIG. 6 IgG1 and IgG2 anti-OVA responses.

FIG. 7 shows IgG1 artibody responses to glycosylated and modified HEL.

FIG. 8 shows immunogenicity of glycoaldehyde-modified OVA.

FIG. 9 snows immunogenicity of glycoaldehyde-modified pigeon cytochrome C.

FIG. 10 shows IgG1 response to glycoaldehyde-modified HEL.

FIG. 11 shows IgG1 response to monomers and homoaggregates of OVA.

FIG. 12 shows interaction of modified HEL with antigen presenting cells.

FIG. 13 shows IgG1 response to a self antigen.

FIG. 14 shows an immune response to a merozoite antigen.

### DETAILED DESCRIPTION OF THE INVENTION

As used herein, the term “antigen” refers to any naturally occurring, recombinant or synthetic product such as polypeptide which is optionally glycosylated, either by production in a cell which naturally produces the polypeptide, or by a heterologous host cell. In the case of the latter, the glycosylation pattern of the polypeptide may differ from that of the naturally occurring polypeptide. The term antigen also includes complexes of protein carriers and non-protein molecules such as steroids, carbohydrates or nucleic acids, wherein the complex is used as an immunogen for the production of an immune response to the non-protein molecule.

As indicated above, we believe that the introduction of an alkyl aldehyde group into an antigen provides substantially enhanced immunogenicity. Any suitable chemical means may be utilized to provide for the introduction of an alkyl aldehyde group. The conditions used will be such that such groups are introduced without adversely affecting the integrity of the antigen. The number of aldehyde groups introduced into an antigen will determined by factors including the reaction conditions used, the nature, if present, of the carbohydrate groups, the structure of the antigen and the like. Introducing from 1 to 4, such as 2 or 3 such groups per antigen molecule is suitable for generating the enhanced immune response observed, but this is not limiting and more groups may be introduced where appropriate.

A suitable method for the introduction of aldehyde groups into a carbohydrate side chain of an antigen is by treatment with periodate. For example, the antigen may be treated in a solution containing from 1 to 100 mM, preferably from 5 to 20 mM periodate (e.g. in the form of its sodium salt). The reaction may additionally be performed in the presence of a buffer to maintain an acidic pH for example in the range of pH 4 to 6.5. For example, the buffer may be phosphate or acetate based buffers (e.g. from 10 to 1000 mM, e.g. 50 to 200 mM), preferably acetate based such as about 50 mM sodium acetate. The amount of antigen treated will vary depending upon its solubility or other considerations. Typically, from 1 to 10 mg/ml of antigen can be treated in the optionally buffered periodate solutions described above.

Alternatively, enzymatic means may be used, such as treatment with glucose oxidase or galactose oxidase. Conditions for the use of such enzymes may be determined by reference to Zheng et al, ibid.

Where an antigen does not have a carbohydrate side chain, we have found that modification of the antigen to introduce such a chain may be effected. For example, the antigen may be treated with a derivatised sugar molecule under conditions suitable to attach the sugar molecule to the antigen. For example, galactopyranosyl-phenylisothiocyanate may be used to introduce galactose residues via the epsilon amino groups of lysine. Suitable conditions include incubation in 0.1 M sodium bicarbonate buffer, pH 9.0, overnight on ice. These conditions may be varied by those of skill in the art using routine knowledge while still providing the desired end product. Other sugars which may be attached include mannan, glucose, galactose, glucuronic acid, glucosamine and other sugars with vicinal hydroxyl groups can be oxidised to form aldehydes. Such sugars may be attached to antigens by analogous means.

Another specific method involves direct modification of a polypeptide by chemical treatment with glycolaldehyde. Generally, a molar excess of glycolaldehyde to protein will be used, for example from 5:1 to 500:1, preferably from 20:1 to 200:1 such as about 100:1. The glycolaldehyde may be incubated with the protein in a buffer at around neutral pH (e.g. from 6.0 to 8.0, such as about 7.4) at above room temperature, (e.g. from 25 to 50° C., such as about 37° C.) for 30 minutes to 5 hours, e.g about 3 hours. The precise conditions may be varied, having regard to the concentrations of reagents used, the nature of the antigen, and the like. The ability of glycol aldehyde to introduce aldehydes into protein antigens is a chemical reactivity which may be shared by other α-hydroxy aldehydes, such as C3-6 α-hydroxy aldehydes, that can be generated through reaction of hydroxy-amino acids with HOCl. Such aldehydes may also be used.

Where a polypeptide antigen which is not normally glycosylated is produced by recombinant means, a carbohydrate side chain may also be introduced by altering the sequence of the polypeptlde so as to introduce a glycosylation site. Generally, N-linked glycosylation occurs at the motif Asn-Xaa-Thr/Ser (where Xaa is any amino acid). A protein sequence may be searched for the presence of a sequence Asn-Xaa²-Xaa³ or Xaa¹-Xaa²-Thr/Ser and the nucleic acid encoding the sequence altered to the glycosylation motif. Such an alteration will be selected to minimise disruption to secondary and tertiary structures of the polypeptide, which may be calculated using algorithms available in the art for this purpose, and/or tested empirically using routine methodology.

The antigen to be modified may be any antigen to which an immune response is required. For example, there is a continuing need to raise polyclonal and monoclonal antibodies in laboratory animals for research purposes. For example, it is often desired to raise such antibodies against newly identified proteins to examine cellular localisation, expression patterns and the like. Antibodies to known antigens are sometimes used for diagnostic purposes, and the methods of the present invention may be used to generate such antibodies in laboratory animals, particularly mice, rats or rabbits. Such antigens include for example cytokines such as TNFα, TNFβ, interlferons α, β, and γ, interleukins IL-1 to IL-18, such as IL-2, IL-6 and IL-12, growth factors such as CSF, GCSF, LIF, and the like.

The antigen may also be an antigen of a pathogenic organism associated with human or animal disease. Organisms which cause animal disease include for example foot and mouth disease virus, Newcastle disease virus, rabies virus and Salmonella species. Organisms which cause human disease include for example bacteria such as Salmonella species including S. typhimurium and S. typhi, Staphylococcus such as S. aureus, Pertussis, Vibrio cholerea, pathogenic E. coli, Mycobacteria species such as M. tuberculosis and M. paratuberculosis. Viral organisms include for example HIV-1 or HIV-2 (which include the viral antigens gp160/120), HBV (which includes surface or core antigens), HAV, HCV, HPV (eg HPV-16), HSV-1 or -2, Epstein Barr virus (EBV), neurotropic virus, adenovirus, cytomegalovirus, polio myelitis virus, and measles virus. Eukaryotic pathogens include yeast, such as C. albicans, amoeba and malaria.

The antigen may also be a tumour associated antigen. Such antigens include CEA, alpha fetal protein (AFP), neu/HER2, polymorphic endothelia mucin (PEM), N-CAM and Lewis Y. The antigen may also be for anti-idiotypic antibodies in combatting B-cell lymphomas.

Antigens such as those mentioned above may be obtained in the form of proteins purified from cultures of the organism, or more preferably by recombinant production of the desired antigen. All of part of the genomes of the above organisms have been cloned and the relevant sequence information is available on publicly available databases, such as Genbank. Methods for the recombinant production of proteins are well known in the art, and such methods may be used for the production of antigens for use in the invention.

Generally, recombinant production of polypeptides will involve cloning DNA encoding the polypeptide of interest into an expression vector, introducing the vector into a suitable host cell, culturing the host cell under conditions to bring about expression of the DNA and production of the polypeptide, and recovering the polypeptide from the culture. Suitable host cells include bacterial cells (such as E. coli), yeast (such as S. cerevisiae or Aspergillus species), or mammalian cells such as CHO or human cells.

Antigens may also be produced by chemical synthesis. Automated peptide synthesisers are commercially available.

Following production of an antigen of the present invention and its treatment to introduce an alkyl aldehyde group, the antigen may be formulated with a pharmaceutically acceptable diluent or carrier, and introduced into a human or animal subject in order to raise antibodies against the antigen. Suitable effluents and carriers will be sterile and pyrogen free, with a suitable isotonicity and stability. Examples include sterile saline (e.g. 0.9% NaCl), water, dextrose, glycerol, ethanol or the like or combinations thereof. The comnostion may further contain auxiliary substances such as wetting agents, emulsifying agents, pH buffering agents or the like. Usually administration will be by the intravenous route, although other routes such as intraperitoneal, subcutaneous, transdermal, oral, nasal, intramuscular or other convenient routes are not excluded.

Doses of any individual antigen will be in accordance with conventional practice in the art, and at the discretion of the physician. Typically, a dose will be in the range of from 1 to 500, such as 5 to 100, for example about 50 μg/kg body weight of recipient.

The dose may be administered in conjunction with an adjuvant, including those mentioned above. A particularly preferred adjuvant is alum (aluminium potassium sulphate dodecahydrate). Alum in the form of a precipitate of aluminum hydroxide (a mixture of AlK(SO₄)₂, H₂O and NaOH is a suitable source of alum available as Imject™ (insoluble AlOH at a concentration of 45 mg/ml, Pierce, Rockford, Ill., USA), used an the accompanying examples. Alum may be used in accordance with manufacturers' instructions. For example, a ratio of from about 100:1 to 500:1 insoluble AlOH:antigen may be used, as illustrated in the accompanying examples, although other forms of alum and other amounts may be used in accordance with standard procedure in the art.

Compositions of the invention comprise a modified peptide of the invention in association with an adjuvant and a pharmaceutically acceptable diluent or carrier. A preferred adjuvant is alum. In one embodiment of this aspect of the invention, the compositions will comprise 1-500 μg, preferably 1-50 μg, of antigen and 0.5 to 20 mg, preferably 1-10 mg, of alum in a pharmaceutically acceptable carrier or diluent as mentioned above. The compositions may be prepared in the form of a concentrate for subsequent dilution, or may be in the form of divided doses ready for administration. Alternatively, the antigen and alum may be provided separately within a kit, for mixing prior to administration to a human or animal subject.

Optionally, the antigen and alum are mixed with one another and incubated for at least 10, preferably at least 30 minutes prior to administration to the subject.

The production and use of antigens according to the invention is particularly advantageous in providing an enhanced IgG1 antibody response to modified antigen which is greater than that observed with unmodified antigen plus alum. This is indicative of a T_{H}2 response. Such enhancement can be observed in the form of an The response may be measured in a suitabie test animal, such as a mouse.

The documents mentioned herein are incorporated by reference. Reference herein to “comprise” or “comprising” and the like is used synonymously with “include” or “including and the like.

The following Examples illustrates the invention.

### Example 1

### Periodace Treatment of Ovalbumin Increases Its Ability to Elicit Antibodies

OVA (5-10 mg/ml) was treated with 10 mM sodium periodate in 0.05 M sodium acetate, pH 4.5, for 90 min at 4° C. (‘standard protocol’). The oxidized OVA was gel filtered through Sephadex G-25 in PBS to remove the sodium metaperiodate.

Groups of CBA/Ca mice (4 per group) were immunized subcutaneously at the base of the tail with 50 micrograms of unmodified OVA in PBS, or with 50, 5, 0.5, and 0.005 micrograms of the periodate-treated OVA in PBS. All mice were boosted on day 28 with 5 micrograms of unmodified OVA in PBS to determine whether immunological memory had been established. The mice were bled at weekly intervals, and the sera were assayed for anti-OVA IgG1 and IgG2a by an ELISA, using plates coated with unmodified antigen and peroxidase-coupled antibodies specific for mouse IgG1 and IgG2a.

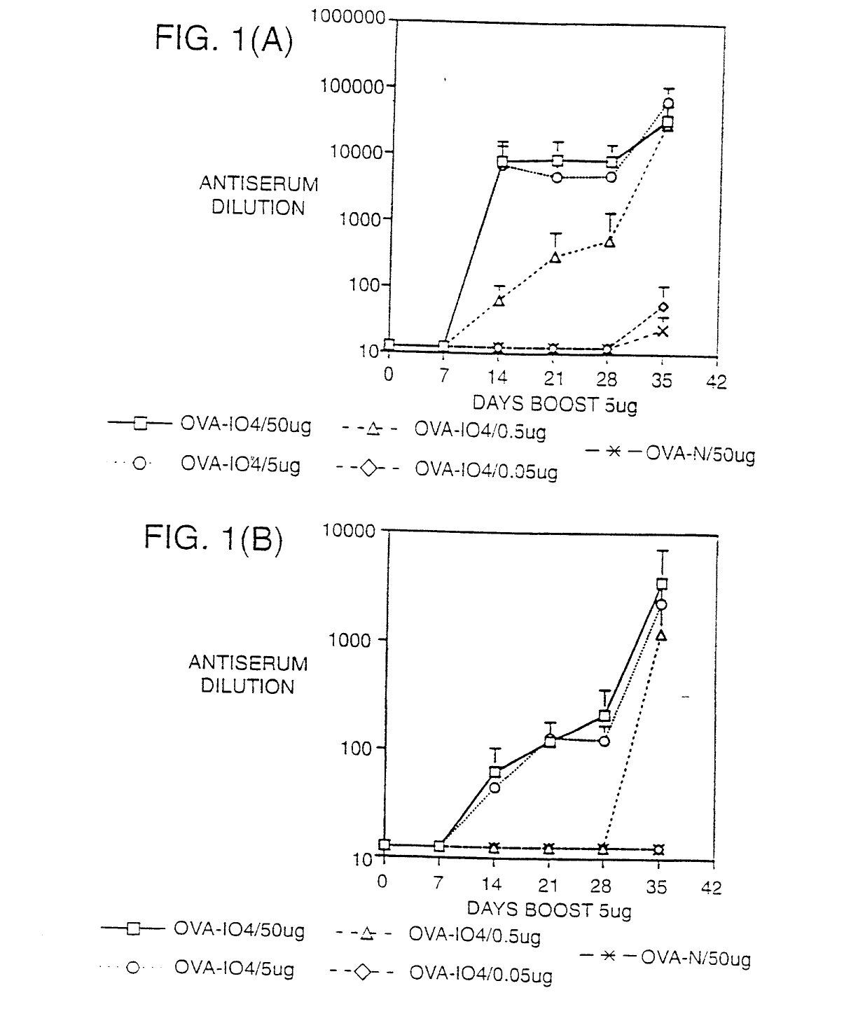

There was no primary resoonse to unmodified OVA, and a memory response could not be induced by the boost. Periodate-modified OVA was highly immunogenic, with as little as 0.5 micrograms causing a primary IgG1 response, and the generatior of memory for unmodified OVA. Lesser amounts of IgG2a were also induced by periodate-modified OVA, but not by unmodified antigen. The results are illustrated in FIG. 1A (IgG1 response) and 1B (IgG2a response).

Antibody titre for mice treated with 50, 5 and 0.5 μg of modified OVA continued to increase when measured at day 56.

Treatment of the periodate modified OVA with 1 mg/ml sodium borohydride for three hours reduced the aldehyde content from 1.9 to 0.2 moles per mole of protein, which resulted in a decrease in immunogenicity of over 90%. Furthermore, it was also found that removal of the N-linked oligosaccharide from OVA by N-glycanase treatment prevented the increase in immunogenicity caused by the subsequent reaction wash periodate.

### Example 2

### The Immune Enhancing Effect of Periodate on Ovalbumin Occurs in Mouse Strains with Different H-2 Haplotypes

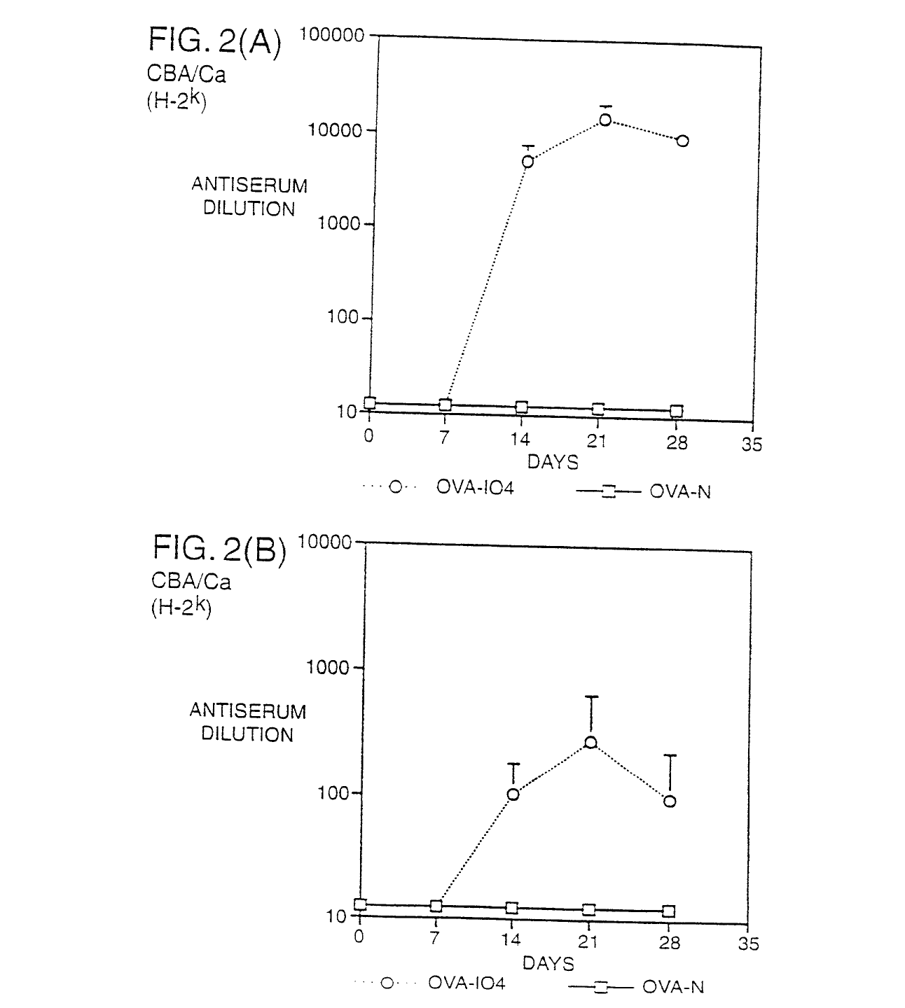

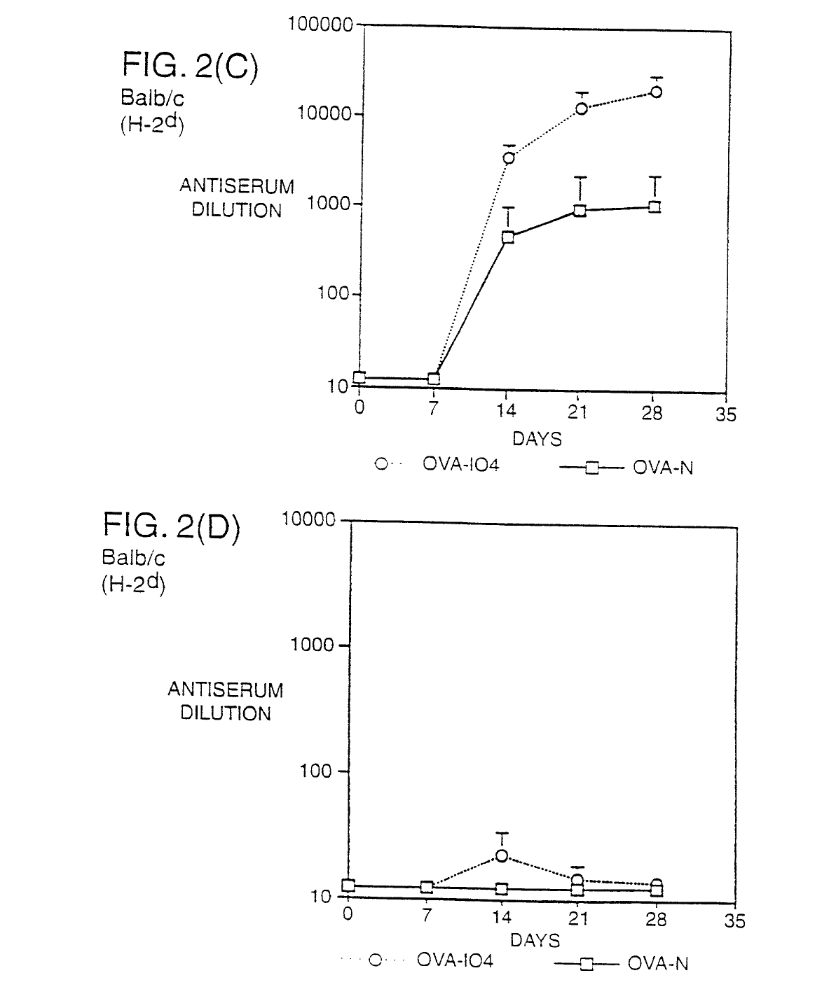

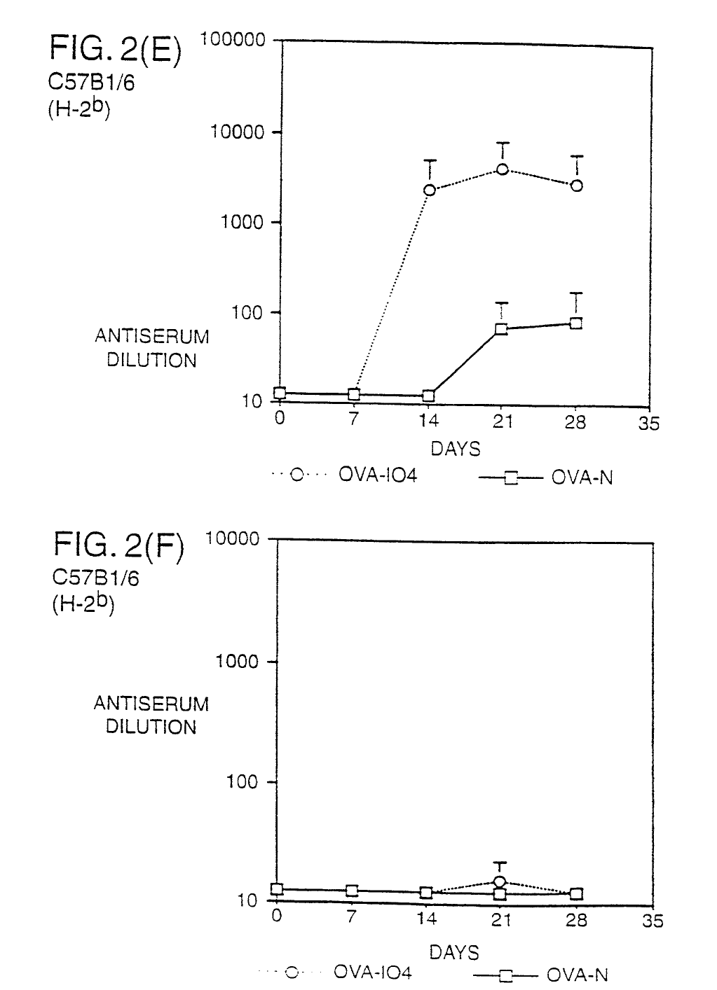

Fifty micrograms of OVA that had been treated with periodate by the standard protocol was administered subcutaneously in PBS to groups of four mice of three different H-2 haplotypes: CBA/CA (H-2^{k}) (FIG. 2A & B), Balb/c (H-2^{d}) (FIG. 2C & D), and C57B1/6 (H-2^{b}) (FIG. 2E & F). They were bled at weekly intervals, and the sera were assayed for anti-OVA IgG1 (FIG. 2A, C & E) and IgG2a (FIG. 2B, D & F).

In each mouse strain, the periodate-modified OVA elicited at least a 10-fold higher IgG1 anti-OVA response than did unmodified OVA. An IgG2a anti-OVA response after primary immunization occurred only with periodate modified OVA in CBA/Ca mice. Therefore, periodate-modified OVA was immunogenic even in the mouse strain most resistant to immunization with unmodified antigen.

Example 3

### Co-immunization with Periodate-Modified Ovalbumin and Hen Egg Lysozyme Does Not Alter the Immunogenicity of the Lysozyme

CBA/Ca mice (4 per group) were immunized subcutaneously with 1 nmol or 0.2 nmol of HEL alone or mixed with 1 nmol of periodate-modified OVA. They were bled at weekly intervals and the sera were assayed for anti-OVA and anci-HEL IgG1.

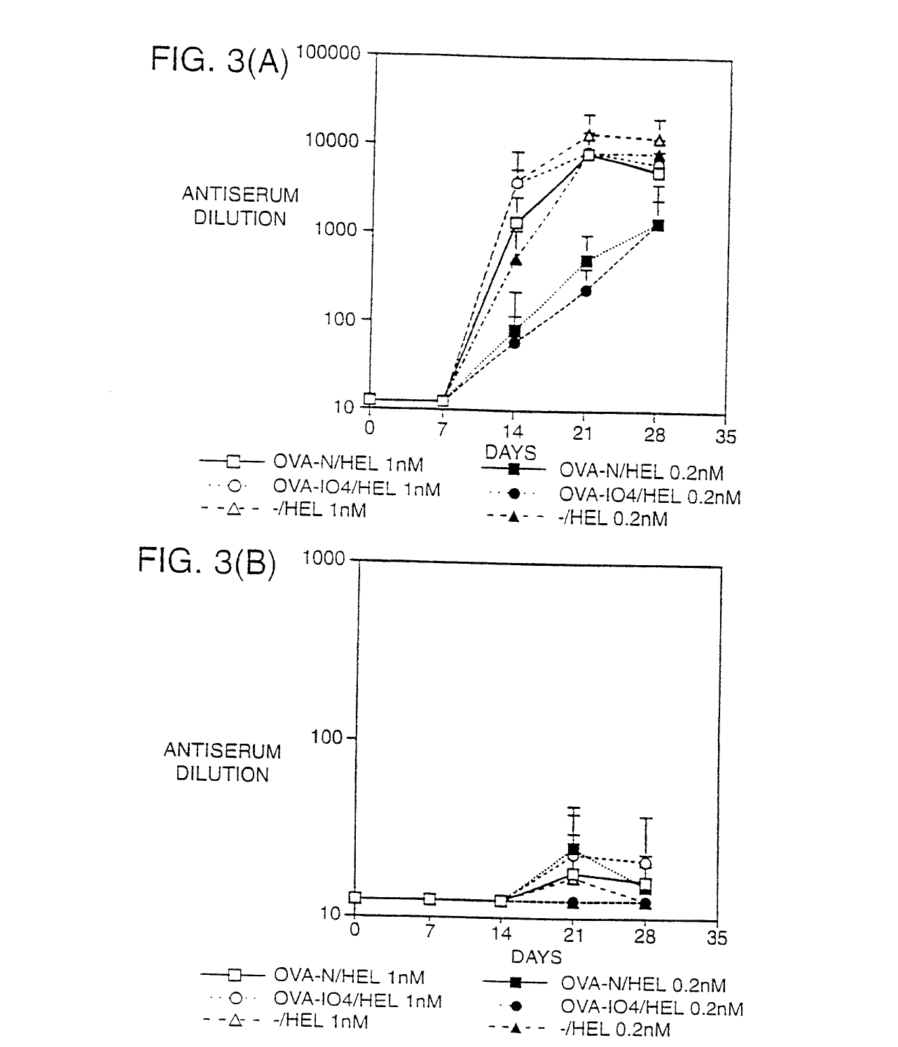

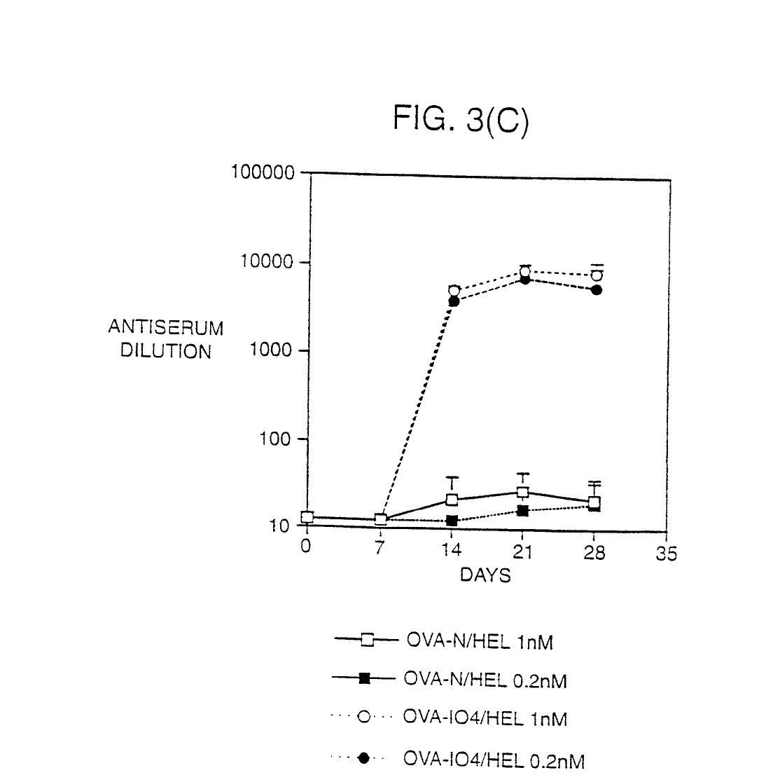

The response to HEL was not enhanced when this antigen was mixed with periodate-modified OVA, although the response to the latter was enhanced. Therefore, the effect of periodate is restricted to the antigen that has been chemically modified. The results are shown in FIG. 3: FIG. 3A—IgG1 anti-HEL response; FIG. 3B—IgG2a anti-HEL response; FIG. 3C—IgG1 anti-OVA response.

### Example 4

### Periodate Treatment of Ovalbumin Increases Its Ability to Elicit Antigen-Scecific Helper T Cells

CBA/Ca mice (4 per group) were immunized subcutaneously with 5 micrograms of unmodified or periodate-modified OVA, or with PBS alone. On day 5, the graining lymph nodes were removed, the lymphocytes were recovered and were incubated with incremental concentrations of unmodified OVA for 64 hrs. During the last 16 hrs, they were pulsed with [³H]-thymidine to measure synthesis of DNA.

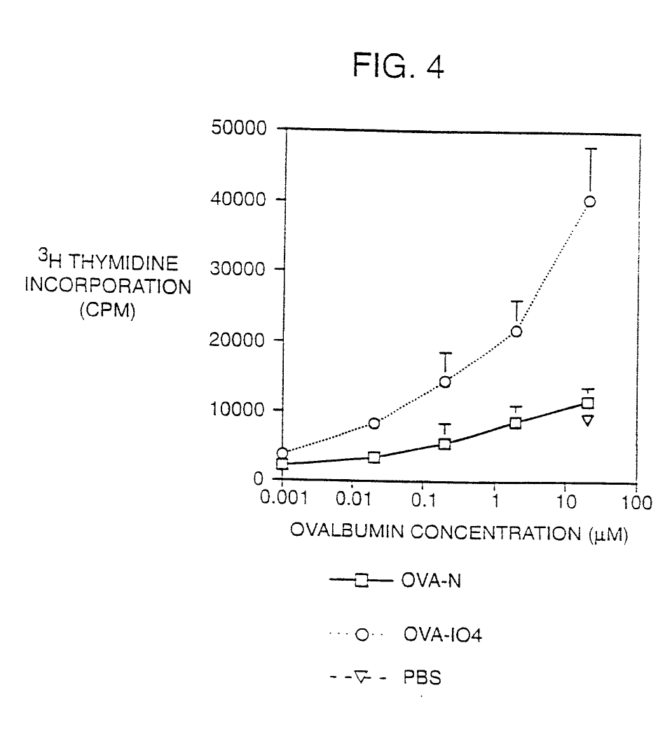

Immunization of mice with periodate-modified OVA, but not with unmodified OVA, caused the generation of antigen-specific T cells that proliferated in response to antigen in vitro. The results are shown in FIG. 4.

### Example 5

### Treatment of Deriodate-Modified Ovalbumin with Borohydride Reduces Its Immunogenicity

OVA that had been treated with periodate was then treated with 1 mg/ml sodium borohydride in 0.1 M sodium bicarbonate for 180 min at 4° C., and then gel filtered to remove the borohydride. Analysis of the antigen for residual aldehydes indicated that the borohydride had reduced these by 90%. CBA/Ca mice (4 per group) were immunized subcutaneously with 5 micrograms of unmodified OVA, periodate-modifri ed OVA, and periodate-, borohydride-treated OVA. Sera were assayed for anti-OVA IgG1 and IgG2a.

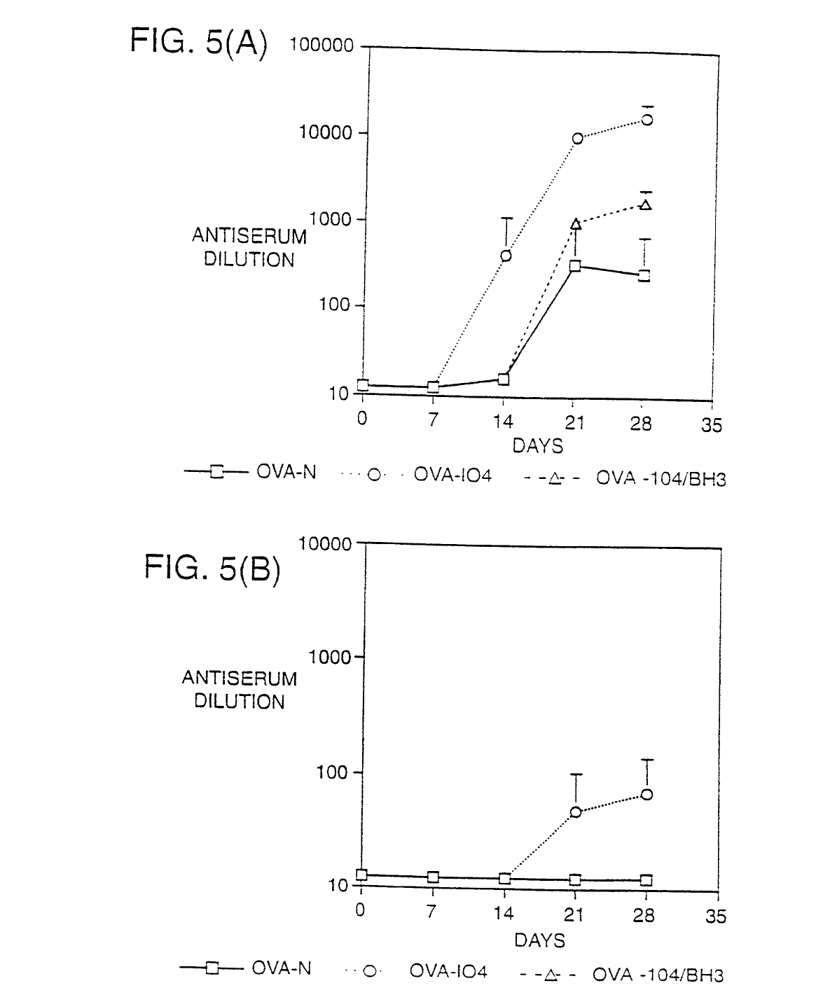

The enhanced responses to periodate-modified OVA were reduced by approximately 90% by treatment of the antigen with sodium borohydride. This finding suggests that it was the generation of aldehydes on the antigen, and not merely the modification of the N-linked oligosaccharide that caused the enhanced immunogenicity. The results are shown in FIG. 5A (IgG1 response) and 5B (IgG2a response).

### Example 6

### N-Deglycosylation of Ovalbumin Prevents the Enhancement of Immunogenicity by Periodate

OVA was deglycosylated with N-glycanase. Portions of deglycosylated and normally glycosylated OVA were treated with periodate, and CBA/Ca mice (4 per group) were immunized subcutaneously with 10 μg of the four forms of OVA. Sera were recovered from the four groups of mice and assayed for anti-OVA IgG1 and IgG2a.

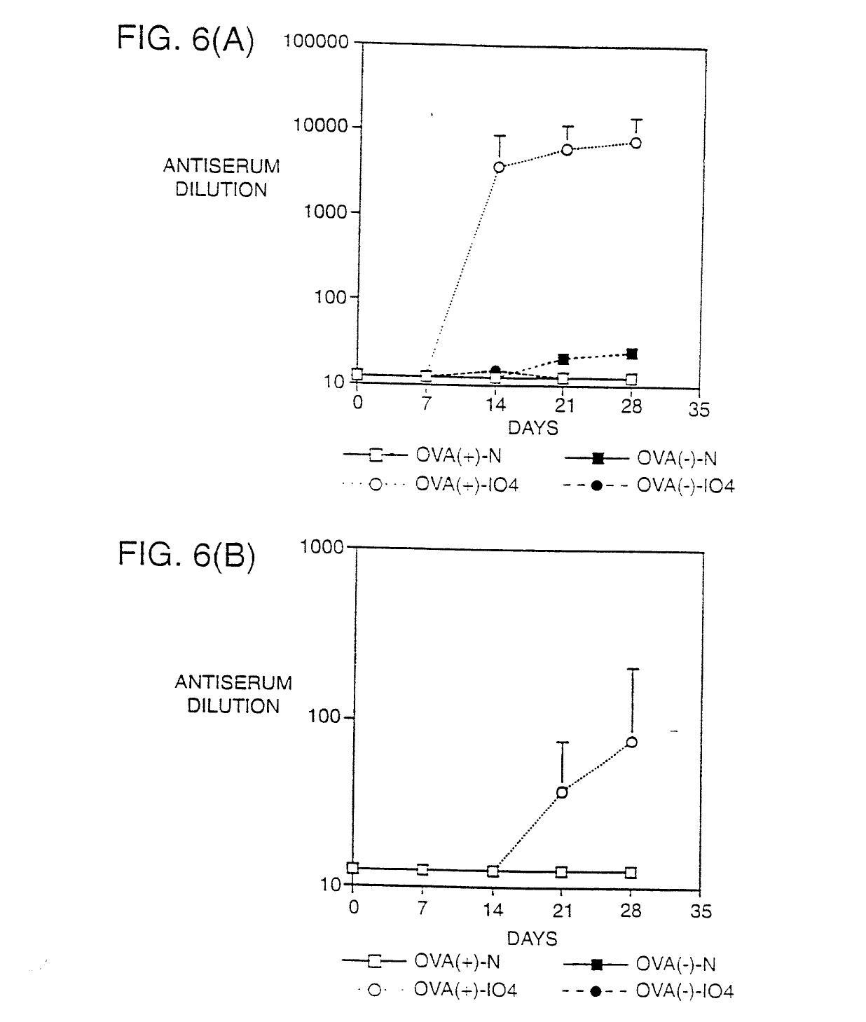

N-deglycosylated OVA alone was not more immunogenic than glycosylated OVA, and treatment of N-deglycosylated OVA with periodate did not enhance its immunogenicity. Therefore, the immune enhancement caused by periodate requires the presence of carbohydrate on the antigen, indicating that it is not mediated by alteration of the protein. Also, simple removal of carbohydrate does not cause a similar enhancement of immunogenicity, indicating that periodate is not mediating its effect merely by destruction of carbohydrate. The results are shown in FIG. 6A (IgG1 antibody titres) and 6B (IgG2a antibody titres).

The treatment with glycanase was as follows: Ovalbumin to be treated with N-Glycanase was run through a Concanavalin A—Sepharose column. The Ovalbumin that did not bind to the column was discarded and the bound Ovalbumin was eluted by addition of excess methyl glucoside to the column. The eluted Ovalbumin was dialysed back into PBS and concentrated. The Ovalbumin was diluted to 2 mg/ml in incubation buffer (20 mM sodium phosphate pH 7.5, 50 MM EDTA, 0.02% (w/v) sodium azide, 0.5% SDS and 5% β-mercaptoethanol). 5% Nonidet P-40 (NP40) was added to make the final NP-40 concentration 1%. 100 Units N-Glycanase (from a 500 U/ml stock) was added and reaction mixture was incubated from 18 hours at 37° C. The solution was then again run through a Concanavalin A—Sepharose column and the protein that did not bind to the lectin (and was therefore de-glycosylated) was collected. Protein was again dialysed into PBS, protein concentration assayed and samples filter sterilised.

One aliquot of the de-glycosylated Ovalbumin was treated with 10 mM sodium periodate as described herein, and another was treated with glycolaldehyde at a 1:100 ovalbumin to glycolaldehyde molar ratio, likewise described herein.

### Example 7

### Attachment of Galactose to A Non-Glycosylated Antigen Allows for Periodate-Mediated Immune Enhancement

HEL, a non-glycosylated protein, was treated with galactopyranosylphenyl isothiocyanate to introduce an average of 1.8 moles of galactose per mole of HEL via attachment to the epsilon amino groups of lysine. CBA/Ca mice (4 per group) were immunized subcutaneously with unmodified HEL, galactose-modified HEL, and galactose-modified HEL that had been treated with periodate. They were bled on day 21 and the sera assayed or anti-HEL IgG1.

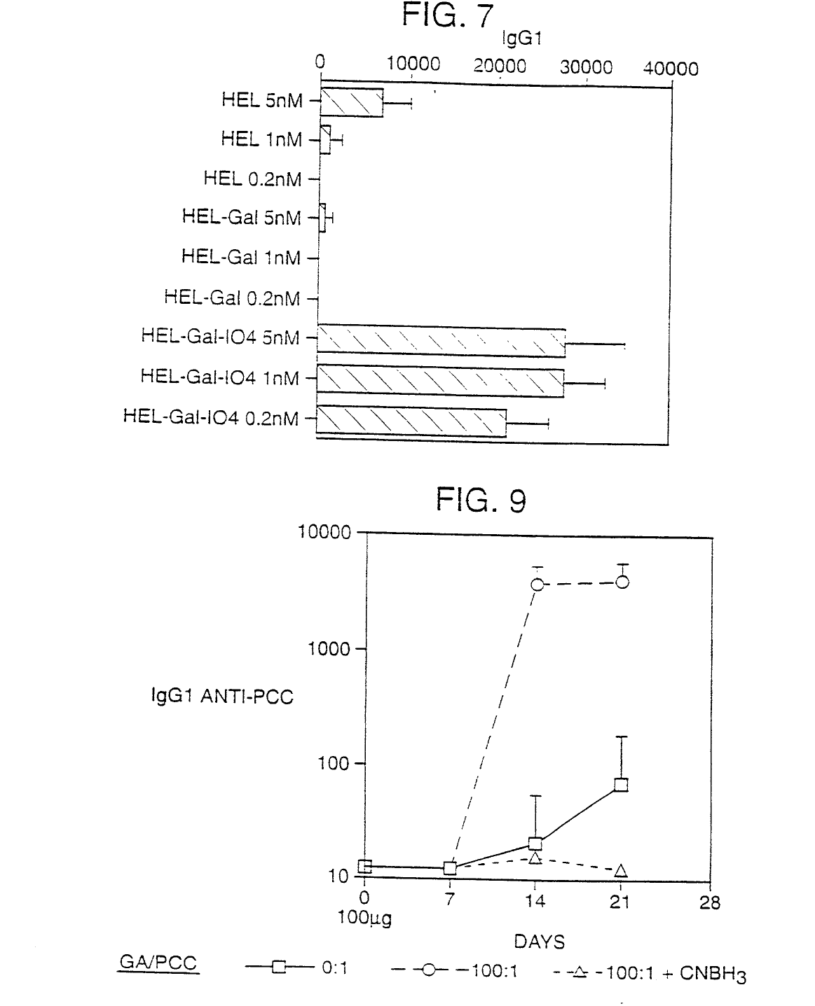

The IgG1 response to HEL was enhanced by sequential galactosylation and periodate modification, and not by galactosylation alone. Therefore, proteins that are not normally glycosylated can have their immunogenicity increased. The results are shown in FIG. 7.

Galactosylazion of HEL was as follows: 15 μl of alpha-D-Galactopyranosylphenylisothiocyanate (α-GPPI), dissolved at 100 mg/ml in Dimethyl Sulphoxide, was added to 2 ml of HEL solution (5 mg/ml in 0.1 M Na₂CO₃). The solution was incubated overnight at 4° C. and dialysed into PBS pH 7.4 (2 exchanges of 2 litres PBS over 6 hours). The protein concentration was calculated and galactose incorporation assayed by Phenol:Sulphuric acid assay.

The assay comprises putting 1 ml of aqueous solution of sample in a colorimetric tube (the solution should contain 10-70 μg of carbohydrate) and adding 1 ml of 5% phenol and mixing. Blanks were prepared by using 1 ml of water instead of carbohydrate-containing solution. 5 ml of 96% sulphuric acid was added rapidly to each tube and shaken. The tubes were incubated for 10 minutes at room temperature then re-shaken, and incubate for 20 minutes in 25-30° C. water bath. Absorbance was measured at 490 nm. The carbohydrate content was calculated by comparison of sample to a standard curve consisting of parallel reactions of galactose solutions with concentrations ranging from 0 to 200 μg/ml.

### Example 8

### Comparative Effects of Periodate, Alum, and Complete Freund's Adjuvant (CFA) on the Immunogenicity of Ovalbumin

CBA/Ca mice (4 per group) were immunized subcutaneously with 50 micrograms of unmodified OVA, periodace-modified OVA, unmodified OVA or periodate-modified OVA adsorbed to alum, and OVA emulsified in CFA. They were bled on day 14, and the sera were assayed for anti-OVA IaG1, IgG2a, and IgG2b.

Anti-OVA Titre (day 14)

| Antigen | IgG1 | IgG2a | IgG2b |
|---|---|---|---|
| Unmodified OVA | 13 ± 2 (2SD) | <12.5 | <12.5 |
| in PBS | | | |
| Unmodified OVA | 1600 ± 927 | <12.5 | <12.5 |
| in alum | | | |
| Periodate-treated | 9153 ± 7997 | 39 ± 35 | 54 ± 33 |
| OVA in PBS | | | |
| Periodate-treated | 21869 ± 8460 | 71 ± 79 | 90 ± 39 |
| OVA in alum | | | |
| Unmodified OVA | 53538 ± 20554 | 890 ± 657 | 1392 ± 1700 |
| in CFA | | | |

Periodate treatment of OVA rendered it more immunogenic than did adsorption to alum. Combining periodate treatment with alum further increased its immunogenicity so that the IgG1 titre was 13-fold greater than that elicited with antigen plus alum alone, and 40% of that induced with antigen in CFA.

Therefore, immunization with periodate-treated antigen combined with adsorption to alum yields an antibody response that is much better than with antigen plus alum alone, which is believed to be the only widely acceptable adjuvant for man, and almost as good as that elicited with antigen plus CFA, the “gold standard” adjuvant which cannot be used in man.

### Example 9

### Glycolaldehyde Treatment of Ovalbumin

Ovalbumin (OVA) was used at final concentration of 50 mg/ml (ie 1.15 mM) in PBS pH 7.4. is was added to reactions from a stock solution of 125 mg/ml. Glycolaldehyde (GA) stock was 1M in PBS pH 7.4. Sodium Cyanoborohydride (Na CNBH₃) stock was 1M in PBS pH 7.4. The follow na reactions were performed:

| GA:OVA Ratio | Vol of OVA | Vol of GA | Vol of NaCNBH₃ |
|---|---|---|---|
| 500:1 | 400 μl | 575 μl | 0 μl |
| 100:1 | 400 μl | 115 μl | 0 μl |
| 20:1 | 400 μl | 23 μl | 0 μl |
| 0:1 | 400 μl | 0 μl | 0 μl |
| 100:1 | 400 μl | 115 μl | 115 μl |

All reactions were made up to a final volume of 1 ml with PBS, briefly vortexed, and left at 37° C. for 3 hours. Reaction solutions were then run through Sephadex G25 columns equilibrated in PBS and 1 ml fractions collected. Fraction(s) containing protein were then dialysed against PBS at 4° C. The samples were filter sterilised through 0.2 μM filters. The samples were used as immunogens on 5 groups of mice, 4 mice per group, as follows:

| | | | |
|---|---|---|---|
| Group 1 | 50 μg | OVA-GA | 1:500 |
| Group 2 | 50 μg | OVA-GA | 1:100 |
| Group 3 | 50 μg | OVA-GA | 1:20 |
| Group 4 | 50 μg | OVA | |
| Group 5 | 50 μg | OVA-GA | 1:100 with CNBH₃ |

All immunisations were given in a total volume of 100 μl in PBS (no adjuvant). immunisations were given subcutaneously into the base of the tail. Weekly tail bleeds were carried out at day 0, day 7, day 14, day 21 and day 28. A boost of 10 μg of the same form of antigen as given at primary immunisation was injected into base of tail at day 32, again in total volume of 100 μl/mouse.

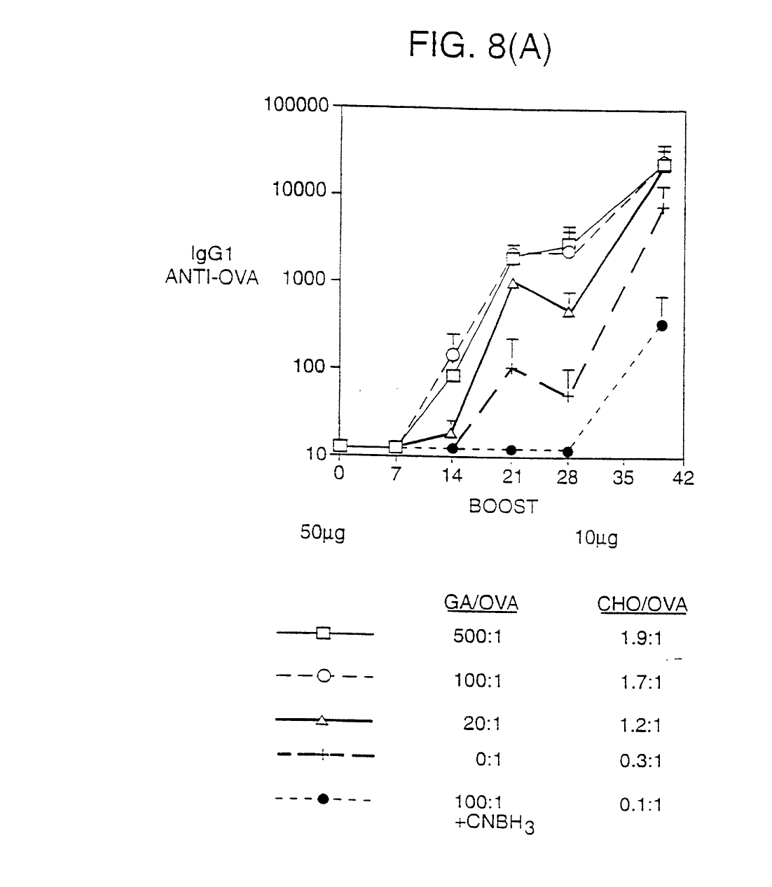

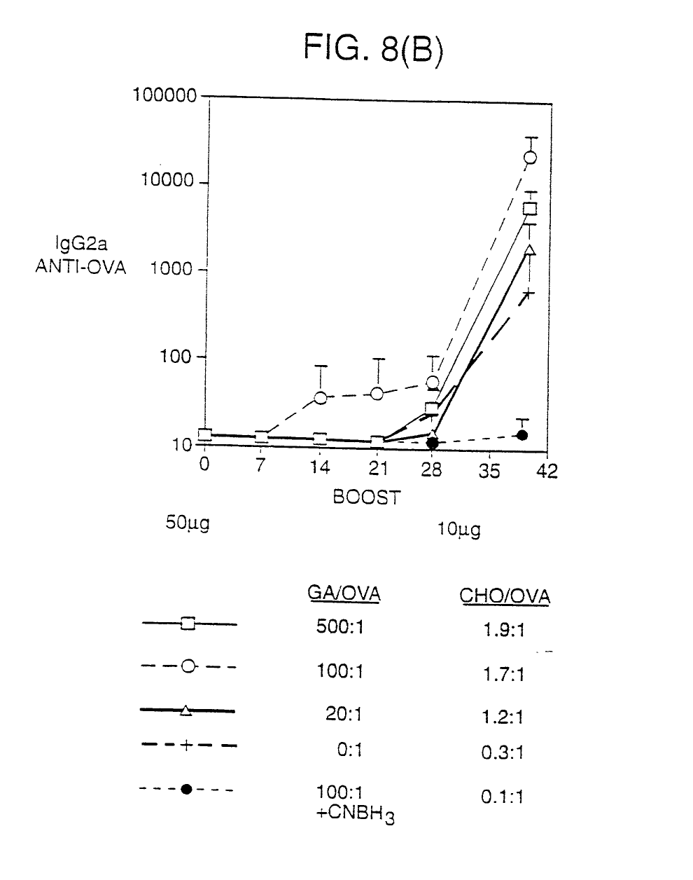

The results are shown in FIG. 8. Glycolaldehyde treatment of OVA result in a boost to the IgG1 (FIG. 8A) and IgG2a (FIG. 8B) responses.

### Example 10

### Glycolaldehyde Treatment of Pigeon Cytochrome C

Pigeon Cytochrome c (PCC) stock was made up to 5 mg/ml (0.41 mM). Glycolaldehyde (GA) stock was 1 M in PBS pH 7.4. Sodium Cyanoborohydride (Na CNBH₃) stock was 1 M in PBS pH 7.4. The following reactions were performed:

| GA:PCC Ratio | Vol of PCC | Vol of GA | Vol of NaCNBH₃ |
|---|---|---|---|
| 0:1 | 500 μl | 0 μl | 0 μl |
| 100:1 | 500 μl | 20.5 μl | 0 μl |
| 100:1 | 500 μl | 20.5 | 20.5 μl |

All reactions were made up to a final volume of 1 ml with PBS, briefly vortexed, and left at 37° C. for 3 hours. Reaction solutions were then run through Sephadex G25 columns equilibrated in PBS and 1 ml fractions collected. Fraction(s) containing protein were then dialysed against PBS at 4° C. The samples were filter sterilised through 0.2 μM filters. The samples were used as immunogens on 3 groups of mice, 4 mice per group, as follows:

| | | | |
|---|---|---|---|
| Group 1 | 100 μg | PCC | |
| Group 2 | 100 μg | PCC-GA | 1:100 |
| Group 3 | 100 μg | PCC-GA | 1:100 with CNBH₃ |

All immunisations were given in total volume of 100 μl in PBS (no adjuvant). Immunisations were given subcutaneously into the base of the tail. Weekly tail bleeds were carried out at day 0, day 7, day 14 and day 21. The results are shown in FIG. 9. Treatment with GA boosts the IgG1 response, and this is reduced by treatment of the PCC-GA with CNBH₃.

### Example 11

### Glycolaldehyde Treatment of HEL

HEL stock was made up at 3 mg/ml and treated at 37° C. for 3 hours in PBS pH 7.4 as described for PCC in Example 10. The modified HEL was then run through a Sephadex G25M column (Pharmacia) pre-equilibriated in PBS and 1 ml fractions were collected. Protein concentration on each of the fractions was performed using Coomassie Blue Plus Protein Reagent against a standard curve composed of BSA. The OD570 was read on a Vₘₐₓ plate-reader and analysed using Softmax software. The protein-containing fractions were then filter-sterilised for immediate use.

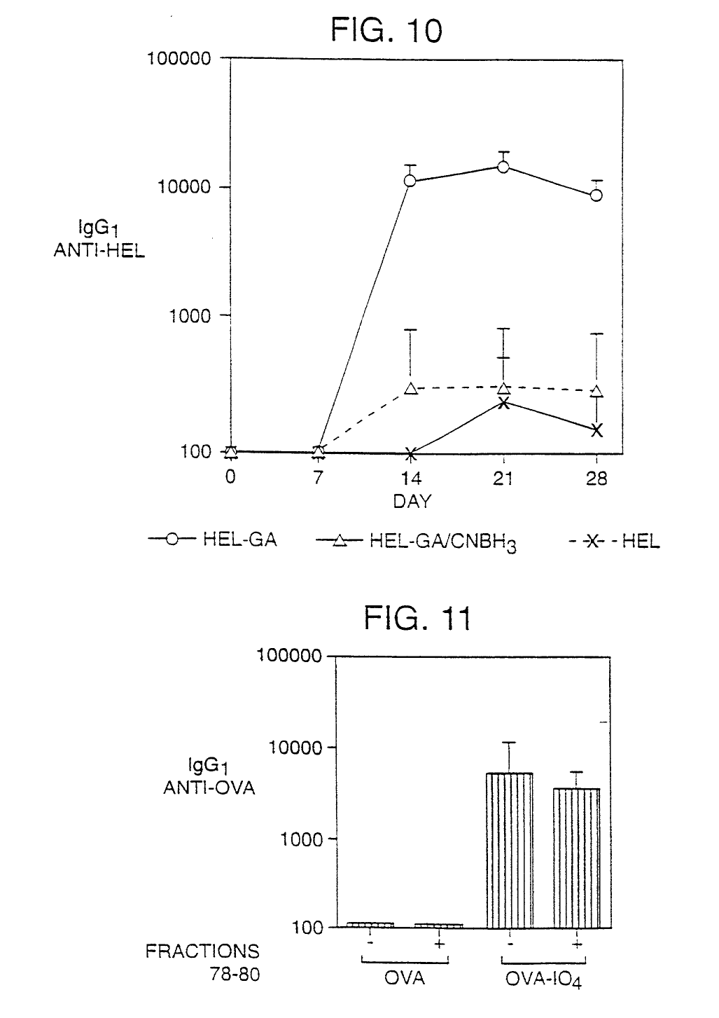

An average of 3 lysines (55%) per mole of HEL were modified in the absence or presence of NaCNBH₃, and 0.9 aldehydes per mole of HEL were generated in the absence of the reducing agent. After 2-3 weeks, the mice that had received unmodified antigens or antigens that had been reacted with glycolaldehyde and NaCNBH₃ had titres of 1/100 or less of IgG1 antibody, reactive with the native forms of the antigens (FIG. 10). In contrast, antibody titres of greater than 1/1000 were present in the mice receiving antigen modified with glycolaldehyde alone.

### Example 12

### Homoaggragate Formation Does Not Enhance Immunogenicity

To determine whether protein aldehydes enhance immunogenicity by mediating the formation of homoaggregates, OVA-IO₄ homoaggregates were prepared. 2 ml of OVA or OVA-IO₄ at 5 mg/ml in PBS was loaded onto a Sephacryl HR100 column (Pharmacia) pre-equilibrated in PBS/0.05% sodium azide and run at a flow rate of 11 mls/hr. Fractions of 1.83 mls were collected using a Pharmacia Superfrac fraction collector. Protein concentration of each fraction was assayed as before and those containing protein were run on 10% SDS/PAGE to confirm their molecular weight. Fractions 78-80, corresponding to the descending limb of the monomeric protein peak, of each form of ovalbumin and were dialysed into PBS and filter sterilised, and mice were immunised. FIG. 11 shows mean OVA-specific IgG₁ titres 14 days after sc immunisation of groups of mice with 50 μg of non-gel-filtered monomeric OVA and OVA-₄ versus 50 μg of the gel-filtered forms, indicating that protein aggregation is not required for immunogenicity.

### Example 13

### Interaction of HEL with Antiaen Presentina Cells

We determined whether the immunogenicity of glycolaldehyde-tagged protein reflected an enhanced interaction with cells capable of antigen presentation.

Modified forms of MEL were prepared as described in Example 11. Murine bone marrow-derived dendritic cells (BMDC were grown in 20 ng/ml GM-CSF. 2×10⁶ BMDC in standard RPMI were incubated on ice with varying concentrations of HEL, glycolaldehyde-modified HEL (HEL-GA) or HEL treated with glycolaldehyde in the presence of cyanoborohydride in a total volume of 200 μl. After 5 minutes the cells were extensively washed with ice-cold PBS and triplicate wells for each sample were established containing 2×10⁵ APC per well. 5×10⁴ 3A9 T cell hybridoma cells (specific for HEL presented on I-A^{K} a kind gift of M S Neuberger) were added to each well and incubated in a total volume of 200 μl/well at 37° C. 50 μl of supernatant was removed from each well after 24 hours and the IL-2 content was assessed by bioassay. The supernatant was added to 1×10⁴ HT2 cells per well and incubated for 16 hours, prior to a ³H-thymidine pulse of 0.5 μCi/well for 5 hours. Incorporated ³H-thymidine was counted on a Beckman scintillation counter.

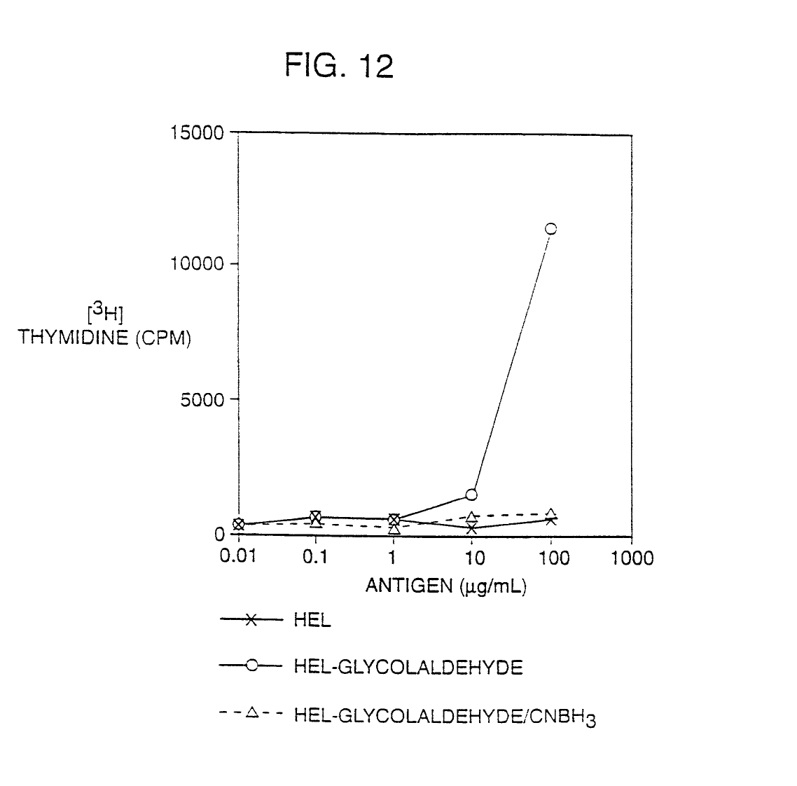

These conditions for interaction or antigen with macrophages were selected to exclude fluid phase endocytosis as the means for antigen uptake. Antigen presentation occurred only with macrophages that had been pulsed with the aldehyde-bearing form of HEL (FIG. 12).

### Example 14

### Alteration of Self-Tolerance

Infection and inflammation have been considered to initiate auto-immunity, either indirectly through a process of antigenic mimicry or directly by enhancing the immunogenicity of self-antigen. The potential of glycolaldehyde for the latter mechanism was examined by immunizing mice with native or modified rat cytochrome C, the sequence of which is identical to the murine protein.

RCC at 2 mg/ml was treated with 10 mM glycolaldehyde with or without the additional presence of sodium cyanoborohydride also at 10 mM for 3 hours at 37° C. in PBS. Samples were run through 10 ml Sephadex G25 columns (Pharmacia) pre-equilibrated in PBS and 1 ml fractions collected and assayed for protein content as before. The protein was filter-sterilised and 100 μg of native, glycolaldehyde-modified RCC and RCC modified by glycolaldehyde in the presence of cyanoborohydride was immunised sc into groups of 4 CBA/Ca mice. Weekly IgG₁ titres against native RCC were assayed by ELISA.

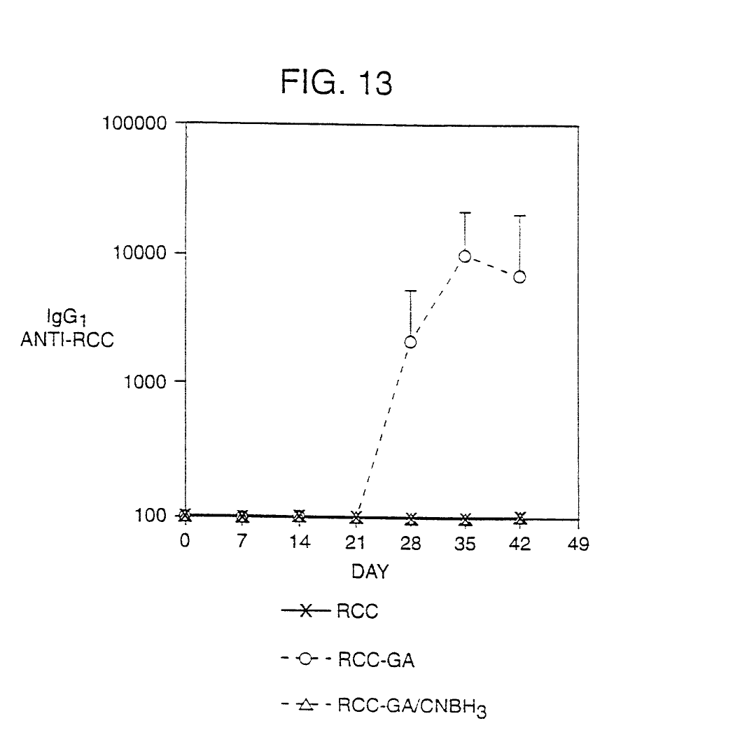

Primary immunisation and a single boost with glycolaldehyde-tagged antigen elicited IgG₁ that was specific for the native self-antigen (FIG. 13). This loss of tolerance did not occur in mice that had been immunised with native antigen or with antigen that had been treated with glycolaldehyde in the presence of NaCNBH₃. Therefore, the alteration of self-antigen by glycolaldehyde can overcome adaptive immune tolerance. Thus the invention may be used to boost immune response to self-antigens in for, example, cancer therapy, particularly therapies where immunization with self-antigens are used, such as in the treatment of melanoma.

### Example 15

### Immune Response to Merozoite Surface Protein

Groups of four mice were immunised subcutaneously with 50 μg in PBS of the 19-kDa carboxyl-terminal fragment of the merozoite surface protein-1 (MSP1) or Plasmodium yoelii expressed in Saccharomyces cerevisiae (Hui, G. S. et al., J. Immunol, 1994, 153 (b), 2544-53). The antigen had been untreated, or reacted with 20 mM glycolaldehyde for 3 hours at 37° C. Groups of mice were also immunised with these antigens in alum. The mice were boosted on day 28 with 20 μg of the same preparations of antigens. Antibody titres to unmodified antigen were measured at weekly intervals.

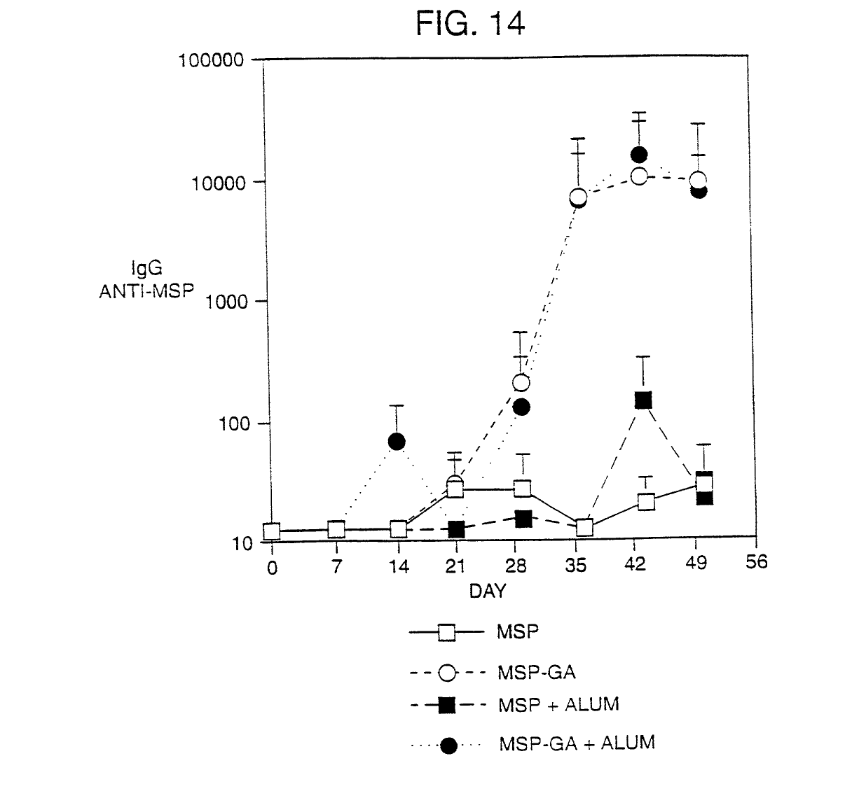

There was no response to unmodified antigen alone or in the presence of alum, whereas a titre of greater than 1/10,000 was observed in mice that had received glycolaldehyde-modified antigen, which was not further augmented by the presence of alum (FIG. 14).

The 19 kd fragment is a candidate protective antigen against Plasmodium yoelii, a murine model for human malaria.

1. A method of producing an immune response in a mammal to an antigen which comprises modifying said antigen by introducing an alkyl aldehyde group into said antigen and introducing said modified antigen into the mammal.

2. A method according to claim 1 wherein said antigen is a glycosylated polypeptide.

3. A method according to claim 1 or 2 which comprises (i) attaching a sugar to said antigen and (ii) modifying said sugar to introduce said alkyl aldehyde group.

4. A method according to claim 2 or 3 wherein said modifying is effected by oxidation with sodium periodate.

5. A method according to claim 1 or 2 wherein said modifying is effected by treatment with glycolaldehyde.

6. A method according to any one of the preceding claims wherein said antigen is introduced into the mammal together with an adjuvant.

7. A method according to any one of the preceding claims in which the antigen is a bacterial, viral, fungal, parasitic or tumour antigen.

8. A composition which comprises (i) an antigen which has been modified by introduction of an alkyl aldehyde group, (ii) an adjuvant, and (iii) a pharmaceutically acceptable diluent or carrier.

9. An antigen which has been modified by introduction of an alkyl aldehyde group for use in a method of treatment of the human or animal body.

10. Use of an antigen which has been modified by introduction of an alkyl aldehyde group for the manufacture or a medicament for immunotherapy of a human or animal subject.
